(19) United States
(12) Patent Application Publication
(10) Pub. No.: US 2022/0208358 A1
(43) Pub. Date: Jun. 30, 2022

(54) SYSTEMS, DEVICES, AND METHODS FOR RAPID DETECTION OF MEDICAL CONDITIONS USING MACHINE LEARNING

(71) Applicant: AVICENNA.AI, Marseille (FR)

(72) Inventor: Cyril DI GRANDI, La Ciotat (FR)

(73) Assignee: AVICENNA.AI, Marseille (FR)

(21) Appl. No.: 17/136,303

(22) Filed: Dec. 29, 2020

(51) Int. Cl. G16H 40/20; G16H 30/20; G16H 30/40; G16H 50/20; G16H 20/40; G16H 15/00; G06N 3/08; G06N 3/04; G06T 7/00; G06K 9/62; G06T 7/11; A61B 5/00

(52) U.S. Cl. CPC G16H 40/20; A61B 5/055; G16H 30/40; G16H 50/20; G16H 20/40; G16H 15/00; G06N 3/08; G06N 3/0454; G06T 7/0012; G06K 9/6227; G06T 7/11; A61B 5/7267; A61B 5/7282; G06T 2207/20081; G06K 2209/051; G06K 2209/27; G06T 2207/20084; G16H 30/20

(57) ABSTRACT

A computing device for processing image data in a medical evaluation workflow is described. The computing device receives image data of a patient and metadata associated with the image data. The computing device analyzes pixel information of the image data using a particular machine learning algorithm to determine one or more acquisition conditions. The computing device selects a machine learning algorithm from a set of machine learning algorithms based on the determined one or more acquisition conditions. The particular machine learning algorithm is different from each machine learning algorithm of the set of machine learning algorithms. The computing device analyzes the pixel information of the image data and the metadata using the selected machine learning algorithm to determine one or more medical conditions of the patient.

### FIELD

The present disclosure relates to systems, devices and methods for prioritizing patients with a medical condition requiring immediate treatment. To this end, the systems, devices and methods utilize pixel data of an image for detection of an acquisition condition.

### BACKGROUND

Intercranial hemorrhages (ICHs) and large vessel occlusions (LVOs) are medical conditions that result in death or permanent catastrophic injuries. For instance, ICHs affect over two million patients worldwide with a 40-50% 1-month patient mortality and 80% disability despite aggressive care. This also results in a major financial burden to healthcare systems, as the average hospitalization cost in the United States is $16,466 for non-survivors and $28,360 for survivors, which equates to $16 billion and $28 billion, respectively. These estimates do not include indirect costs from necessary follow-up diagnostic imaging, medication, or loss of income/productivity from the individuals and their caregivers. While improvements in primary prevention have slightly decreased the incidence of ICHs, the overall mortality remains unchanged.

For a patient with an untreated LVO (e.g., a patient having a stroke), every minute results in the loss of over 2 million neurons, 14 billion synapses, and 12 km of myelinated fiber. As a result, LVOs are associated with a 4.5× increased odds of death and a 3.0× increased odds of a poor outcome for those who survive.

Timely rapid and accurate diagnosis of these medical conditions is necessary to permit immediate treatment so as to prevent death and catastrophic injuries. For instance, rapid identification of ICH patients would facilitate immediate control of blood pressure during the vulnerable first few hours of symptom onset where acute deterioration is most likely.

Currently, report times for neuro-critical findings on non-contrast computed tomography (NCCT) head examinations, which is used to diagnose these medical conditions, can range from 1.5-4 hours in the emergency room setting. In addition, 16% of critical findings are never reported to referring clinicians. Such delays impact patient care as acute deterioration from hemorrhagic expansion often occurs early—within the initial 3-4.5 hours of symptom onset.

Current systems aimed at providing quick diagnoses of these medical conditions involve analyzing metadata information in non-image headers of the patients' medical images. However, use of this metadata information has proven to be unreliable in providing an accurate diagnosis.

Therefore, systems, devices, and methods for sorting and prioritizing patients that require immediate medical attention are needed to reduce the fatality rate of such patients.

### SUMMARY

The present disclosure describes systems, devices, and methods that meet this challenge by processing pixel data of an image for detection of acquisition conditions to facilitate the selection of appropriate algorithms for providing clinicians with real-time alerts prioritizing patients that likely require immediate medical attention.

A computing device for processing image data in a medical evaluation workflow is disclosed. The computing device may include a processor and a memory where the memory stores instructions that, when executed by the processor, cause the computing device to perform the following steps. The computing device receives image data of a patient and metadata associated with the image data. The computing device analyzes pixel information of the image data using a particular machine learning algorithm to determine one or more acquisition conditions. The computing device selects one or more machine learning algorithms from a set of machine learning algorithms, based on the determined one or more acquisition conditions. The machine learning algorithms of the set of selectable machine learning algorithms analyze the pixel information of the image data and the metadata to determine one or more medical conditions of the patient.

A method for processing image data in a medical evaluation workflow is disclosed. The method may include receiving, by a computing device, image data of a patient and metadata associated with the image data. The method may include analyzing, by the computing device, pixel information of the image data using a particular machine learning algorithm to determine one or more acquisition conditions. The method may include selecting, by the computing device, one or more machine learning algorithms from a set of machine learning algorithms based on the determined one or more acquisition conditions. The machine learning algorithms of the set of selectable machine learning algorithms analyze the pixel information of the image data and the metadata to determine one or more medical conditions of the patient.

### BRIEF DESCRIPTION OF THE DRAWING FIGURES

The scope of the present disclosure is best understood from the following detailed description of exemplary embodiments when read in conjunction with the accompanying drawings. Included in the drawings are the following figures:

FIG. 1 depicts an example system for determining medical conditions of a patient in accordance with illustrative embodiments of the present disclosure.

FIG. 2 depicts an illustrative flow diagram for determining medical conditions of a patient in accordance with illustrative embodiments of the present disclosure.

FIG. 3 illustrates an exemplary screen-shot of an application that displays pop-up notifications of new studies with suspected findings.

FIG. 4 illustrates an exemplary screen-shot of a pop-up containing patient name, accession number and the type of suspected findings.

FIG. 5 shows exemplary image data in the form of NCCT images.

Further areas of applicability of the present disclosure will become apparent from the detailed description provided hereinafter. It should be understood that the detailed description of exemplary embodiments are intended for illustrative purposes only and, therefore, are not intended to limit the scope of the disclosure.

### DETAILED DESCRIPTION

Illustrative embodiments of the present disclosure provide a manner of sorting and prioritizing patients with a medical condition requiring immediate attention. Particularly, image data of a patient resulting from a medical scan may be analyzed using an initial machine learning algorithm to determine one or more acquisition conditions. The image data may be examined to the exclusion of any metadata corresponding to the image data in order to avoid negative effects caused by possibly unreliable metadata. Based on the acquisition conditions, one or more different machine learning algorithms may then be selected. The image data and its corresponding metadata may be analyzed based on the different machine learning algorithm to determine one or more potential medical conditions of the patient.

Different acquisition conditions may be associated with various machine learning algorithms. As a result, the initial machine learning algorithm operates to determine the most appropriate machine learning algorithm for analyzing the image data and metadata to accurately determine the medical conditions of the patient. Because the initial machine learning algorithm does not rely on the metadata, accuracy is improved since metadata may, in some cases, be unreliable.

The systems, devices, and methods can be used for analyzing non-enhanced head computed tomography (CT) images and/or CT angiographies of the head, which can assist healthcare providers in workflow triage by flagging and communicating suspected positive findings of, for example, head CT images for ICHs and CT angiographies of the head for LVOs. This allows for expedited intervention and treatment of the medical conditions.

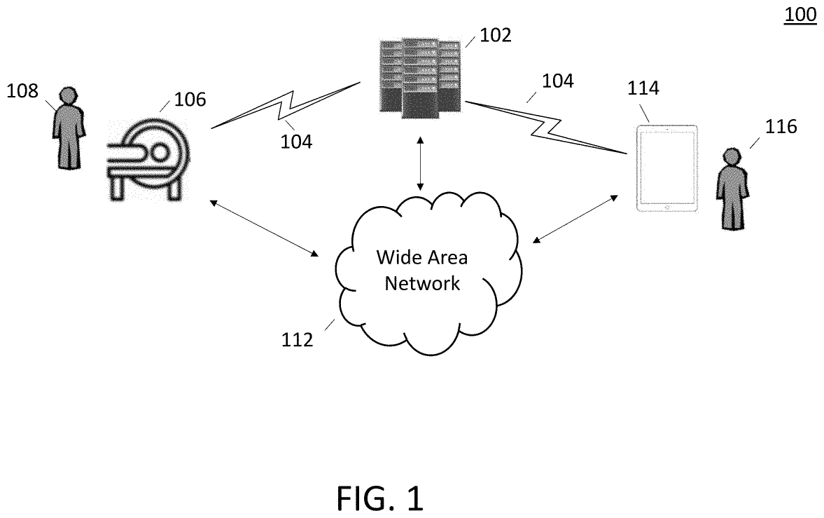

FIG. 1 depicts an example system for determining medical conditions of a patient in accordance with illustrative embodiments of the present disclosure. The system 100 includes a triage server 102, one or more imaging modalities 106, a doctor computing device 114, which may be communicatively connected via an internal or local network 104 and/or a wide area network 112.

The imaging modalities 106 may be responsible for generating image data and metadata corresponding to the image data for a patient. The imaging modalities 102 can include computed tomography (CT), magnetic resonance imaging (MRI), Ultrasound, and X-ray procedures, or other such medical imaging procedures. The imaging modalities 102 can use an energy source such as x-rays or magnetic fields to capture the image data of a subject (e.g., a patient). The imaging modalities 106 may be controlled by an operator 108 (e.g., a nurse, doctor, technician, etc.) at a medical facility through the use of a workstation terminal or other electronic input control. The technician conducting the imaging procedure for a patient may enter information into the electronic input control.

The image data may include one or more images of one or more body parts of the patient. The image data can be in a Digital Imaging and Communications in Medicine (DICOM) standard, other industry-accepted standards, or proprietary standards. The image data can be a visual representation of the interior of a body for clinical analysis and medical intervention, as well as visual representation of the function of some organs or tissues (physiology).

The imaging modality 106 itself and/or computer (not shown) communicatively connected to the imaging modality 106 generates the metadata for the image data. The metadata may include patient information (e.g., name, age, medical history, symptoms or reasons for scan, etc.), timing information (date, time, etc.), and the like. In one example use case, a patient may undergo a head-neck MRI with contrast agent injection on suspicion of cerebral vascular accident. The metadata may indicate “head-to-neck study with contrast agent” and/or risk of stroke. In some instances, the metadata may be very scant such as “H/N−W” to describe the situation. The scant notation of this metadata may cause errors as it omits information to adequately describe the image data. In some cases, some of the metadata may be entered, by the operator 108, via a man-machine interface (MMI) of the imaging modality 106 or a computer connected to the imaging modality 106.

While the imaging modalities 106 are described as separate devices from the triage server 102, in one or more arrangements, the imaging modalities 106 may be part of the triage server 102.

The triage server 102 may be responsible for analyzing received image data and metadata of a patient and determining medical conditions of the patient. As will explained further detail below, the triage server 102 may be configured to receive image data and metadata. The triage server 102 may analyze the image data using a machine learning algorithm to determine one or more acquisition conditions. The metadata may be excluded in this examination in order to avoid tainting of the results of the analysis by possibly unreliable metadata. Based on the acquisition conditions (described in detail below), a second machine learning algorithm may then be selected. The image data and its corresponding metadata may be analyzed based on the second machine learning algorithm to determine one or more medical conditions of the patient.

The triage server 102 may include one or more databases, which may be relational or non-relational databases. For instance, the triage server 102 may include a first database that maps acquisition conditions to machine learning algorithms. As an example, a first acquisition condition may be mapped to one or more machine learning algorithms and a second acquisition condition may be mapped to one or more different machine learning algorithms. The triage server 102 may include a second database that maps possible results of machine learning algorithms to potential medical conditions. As an example, a first result may be mapped to one or more medical conditions and a second result may be mapped to one or more different medical conditions. The triage server 102 may transmit notifications of the patient's one or more medical conditions to the doctor's computing device 114 for display to the patient's doctor 116. The doctor's computing device 114 may be, for example, a tablet, smartphone, personal computer, laptop, workstation, etc. Additionally or alternatively, the database of the triage server 102 includes event rules to select and trigger one or several machine learning algorithms based on detected acquisition conditions, which is described in additional detail below.

The triage server 102 can also receive information from external nodes 104 such as a computer/server storing electronic medical record (EMR) or healthcare information system (HIS), which may be used to aid in the determination of the one more medical conditions.

Data communications between the imaging modalities 106 and the triage server 102 and/or between the processing server 102 and the doctor's computing device 225 may be transmitted over internal or local networks 104 or wide area network 112. Internal or local networks may be a network specific to a medical facility such as a hospital or doctor's office. Wide area networks may include one or more of satellite networks, cellular networks, Internet, etc.

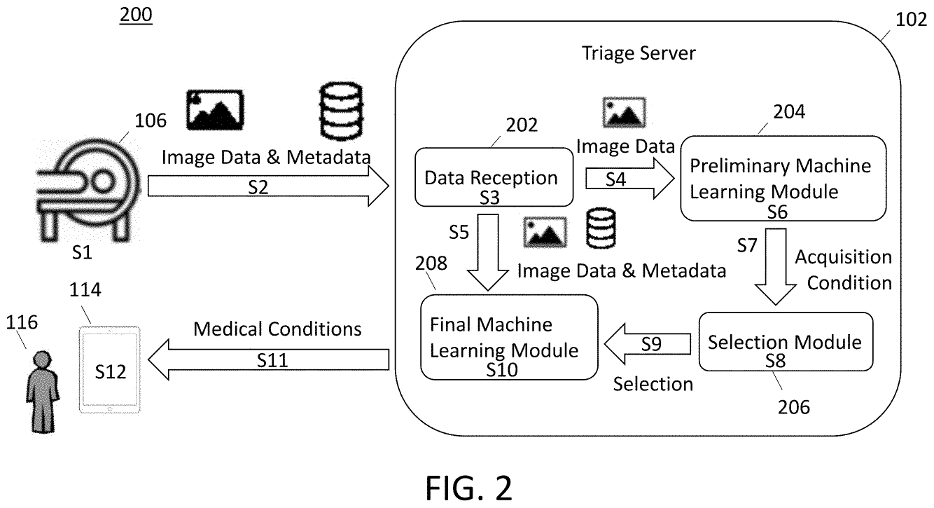

FIG. 2 depicts an illustrative flow diagram 200 for determining suspected medical conditions of a patient in accordance with illustrative embodiments of the present disclosure. The flow may begin at step S1 in which image data of a patient's body part may be captured by an imaging modality 106. Additionally, in step S1, metadata associated with the image data may be generated. Some of the metadata may be generated by the imaging modality 106 that captured the patient's image data. This metadata may include, for example, a timestamp of the image, current settings of the imaging modality 106, the name of the medical professional that operated the imaging modality 106 to capture the image data, body part identifier, hardware manufacturer/model of device that produced image data 105, motion artifacts, and the like. Some of the metadata may be entered by a medical professional or received from a health record database. This metadata may include, for example, the patient's name, age, height, weight, gender, medical history, other characteristics specific to the patient, and name of the patient's physician 116. The metadata may be included in non-image headers of the image data.

In some instances, the metadata include data that may be unreliable since the metadata may be erroneous, incomplete, or uninterpretable. Typically, prior art systems that sort patients rely only on metadata defined by the medical device or operator. In instances where the metadata is unreliable, prior art systems may use an irrelevant algorithm to analyze the patient's information leading to omission of the patient's actual medical conditions and/or false-positives of other medical conditions. Further, by only analyzing the metadata, prior art systems under-interpret the patient's image data. The systems, devices, and methods of the present disclosure do not have such issues since, as will be explained below, acquisition conditions are determined using pixel data of the patient's images and not the metadata of the patient's images. Examples of unreliable metadata my include erroneous information entered by the operator, shorthand notations entered by the operator, metadata descriptions not identifying all of the different organs visible within the images, and the like.

At step S2, the imaging modality 106 may transmit the image data and corresponding metadata to a data reception module 202 of the triage server 102. The triage server 102 may also include a preliminary machine learning module 204, a selection module 206, and a final machine learning module 208. At step S3, a data reception module 202 of the triage server 102 may receive the image data and metadata and determine how the data is to be handled. The data reception module 110 can be configured to examine the image data. The process of examining/validating the image data can include assessing data parameters of the quality, size, and/or format of the image data. Examining/validating the image data can provide certain well-defined guarantees for fitness, accuracy, and consistency for any type data into an application or automated system. Data validation rules can be defined and designed using any of various methodologies and can be deployed in any of various contexts. Image data that does not conform to data parameters can be filtered out (e.g. filtering incomplete/low quality/inappropriate physiology data) or can be modified within certain limits by compressing (e.g. compressing the image data to conform to size standards) and/or converting (e.g. converting the image data to a DICOM standard). The data reception module 106 can also store the image data on a storage device for further access and processing. In instances where the image data does not satisfy one or more of the above quality standards, the triage server 102 may send a notification to one or more of the physician's device 114, the imaging modality 106, a computer of a medical professional who operated the imaging modality 106 to capture the image data, etc.

At step S4, the data reception module 202 may transmit image data without metadata to the preliminary machine learning module 204. If the metadata is part of the non-image header of the image data, the data reception module 202 may separate the non-image header from the remaining image data and then send only the remaining image data to the preliminary machine learning module 204. As a result, the preliminary machine learning module 204 does not receive any metadata and performs its analysis using pixel information of the image data.

At step S6, the preliminary machine learning module 204 may analyze image data (e.g., the pixel data) using an initial learning algorithm to determine one or more acquisition conditions. Examples of the initial learning algorithm include deep learning algorithms such as classification algorithms and/or segmentation algorithms based on convolution neuronal networks (CNN). In some cases, the initial learning algorithm may be a multi-class segmentation algorithm in order to deal with several classes at the same time (e.g., brain, lung, etc.). The acquisition conditions may be determined characteristics of the pixel data. The acquisition conditions indicate the kind of data that has been received. An example of an acquisition condition includes identification of at least a part of a particular organ depicted in the image data (e.g., left side of the heart, right side of the brain, lower femur, etc.). An example of an acquisition condition may be an image artifact (e.g., streak, motion blur, etc.). Another example of an acquisition condition may include detection of a piece of non-biological material depicted in the image data (e.g., a metal plate or rod, etc.). Other examples of acquisition conditions may include a type of reconstruction, presence of contrast or contrast phase in imaged blood vessels in the image data, pre-surgery conditions, post-surgery conditions (e.g., detection of surgical threads or wires), etc.

The initial deep learning algorithm may provide a combination of acquisition conditions. As an example, a combination of acquisition conditions may include non-contrast head CT, soft tissue reconstruction, not Post op, and axial acquisition. As another example, a combination of acquisition conditions may include thorax CT angiography mediastinum reconstruction and not Post op. As another example, a combination of acquisition conditions may include non-contrast cervical spine CT and bone reconstruction. As yet another example, the acquisition conditions may include MR Diffusion Weighted Imaging of the Head. As yet another example, the acquisition conditions may include thorax/pelvis/abdomen CT soft tissue reconstruction.

At step S7, the acquisition conditions determined by the preliminary machine learning module 204 are sent to the selection module 206. At step S8, the selection module 206 may select, based on the received acquisition conditions, one or more machine learning algorithms to apply in order to determine the one or more medical conditions of the patient. These selected machine learning algorithms are different from the preliminary machine learning algorithm. As discussed above, the triage server 102 may include a database that maps acquisition conditions to machine learning algorithms. As an example, a first acquisition condition, or set of acquisition conditions, may be mapped to one or more machine learning algorithms, and a second acquisition condition, or set of acquisition conditions, may be mapped to one or more different machine learning algorithms. The machine learning algorithms listed in the database are different from the preliminary machine learning algorithm.

Additionally or alternatively, the database of the triage server 102 includes event rules to select and trigger one or several machine learning algorithms based on detected acquisition conditions. The selection module 206 may select one or several machine learning algorithms based on the event rules and the determined one or more acquisition conditions.

In one example use case, a patient may undergo a head-neck MRI with contrast agent injection on suspicion of cerebral vascular accident. The metadata may indicate “head-to-neck study with contrast agent” and/or risk of stroke. In some instances, the metadata may be very scant such as “H/N−W” to describe the situation. By analyzing the pixel data (and not the metadata) in S6, the preliminary machine learning module 204 may determine that the patient underwent a head-neck MRI with contrast agent injection. The preliminary machine learning module 204 also identifies the different organs visible on the images (e.g., part of the lung). The preliminary machine learning module 204 may identify one or more of the following acquisition conditions: CT, contains brain, contains head, contains neck, contains lung, with contrast agent, without contrast agent, soft tissue reconstruction, etc.

Examples of selectable machine learning algorithms include large vessel occlusion detection, intracranial hemorrhage detection, pulmonary embolism detection, Alberta stroke program early CT score (ASPECTS), etc. ASPECTS is a 10-point quantitative topographic CT scan score used in patients with middle cerebral artery (MCA) stroke. Segmental assessment of the middle cerebral artery (MCA) vascular territory is made and 1 point is deducted from the initial score of 10 for every region involved. The event rules may cause selection of one or more of the algorithms based on the acquisition conditions. As an example, if the acquisition conditions include “contains head” and “with contrast agent,” then the large vessel occlusion detection algorithm may be selected to detect ischemic stroke. As another example, if the acquisition conditions include “contains lung” and “with contrast agent,” then pulmonary embolism detection algorithm may be selected.

In some instances, the event rules may use output of one of the selectable machine learning algorithms as a basis to select another selectable machine selectable algorithm. As an example, if the acquisition conditions include “contains head” and “without contrast agent,” then the intracranial hemorrhage detection algorithm may be selected. If the result of the intracranial hemorrhage detection algorithm indicates that a hemorrhage was detected, then hemorrhage may be identified as the medical condition. However, if the result of the intracranial hemorrhage detection algorithm indicates that a hemorrhage was not detected, this may serve as the basis to select another selectable machine learning algorithm. For instance, if the acquisition conditions include “contains head” and “without contrast agent” and the output of the intracranial hemorrhage detection algorithm indicates that a hemorrhage was not detected, then the ASPECTS score algorithm may be selected.

At step S9, the selection module 206 provides an indication of the selected machine learning algorithms to the final machine learning module 208. At step S5, the data reception module 202 sends the image data and the metadata to the final machine learning module 208.

At step S10, the final machine learning module 208 may use the selected machine learning algorithms to analyze both the image data and the metadata to determine one or more medical conditions (e.g., LVO, ICH) of the patient. The final machine learning module 208 may include multiple machine learning algorithms and may select the appropriate machine learning algorithms based on the indication received from the selection module 206. An example of a machine learning algorithm includes machine learning algorithms that detect suspected findings of an ICH (Intracranial hemorrhage). Another example of a machine learning algorithm includes automatic segmentation of the prostate and volume computation. Another example of a machine learning algorithm includes automatic Prostate Imaging-Reporting and Data System (PiRADS) computation on a multi parametric MR Acquisition including diffusion and T1 weighted imaging. Yet another example of a machine learning algorithm includes automatic segmentation of the infarct core (e.g., Stroke) on a head CT perfusion acquisition.

Unlike the preliminary machine learning module 204, the final machine learning module 208 analyzes both image data (e.g., pixel information) and metadata. However, because the machine learning algorithm being used by the final machine learning mobile 208 was selected without analyzing the metadata, the selected machine learning algorithm is the most appropriate machine learning algorithm to determine correct medical conditions even if the metadata may be unreliable.

In one example use case, the acquisition condition may be a non-contrast head CT. In such a use case, the selected machine learning algorithms may include three machine learning algorithms (e.g., intracranial hemorrhage detection, midline shift detection, and hydrocephalus evaluation).

In another example use case, the acquisition conditions may include thorax and head CT angiography (e.g., not a fully thorax acquisition but starts around the heart). In such a use case, the selected machine learning algorithms may include large vessel occlusion detection (brain vessels application), pulmonary embolism detection (lung vessels application), and automatic stenosis evaluation (neck vessels application).

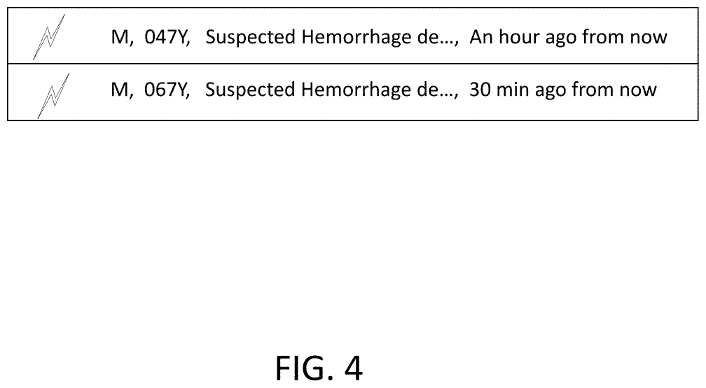

Based on detections of suspected medical conditions, the triage server 102 may modify electronic workflow. As an example, the triage server 102 may, at step S11, transmit a notification to a remote computing device (e.g., a physician's device) such that, at step S12, a pop up may be displayed to the physician to inform the physician of the suspected medical condition for a particular patient (see e.g., FIG. 4). As another example, the triage server 102 may automatically modify a schedule to the physician by prioritizing patients with suspected medical conditions (e.g., LVO, ICH) over those without suspected medical conditions. The updated schedule may also prioritize those patients that were diagnosed with suspected medical conditions over later diagnosed patients with suspected medical conditions.

Additionally or alternatively to outputting a notification, the triage server 102 may perform other actions. As an example, the triage server 102 may generate or update a worklist to highlight urgent situations (e.g., upgrade a worklist of a radiologist). For instance, the triage server 102 may determine a priority for the patient based on a determination of patient's possible medical conditions and update a worklist (e.g., the list depicted in FIG. 4) based on the determined priority. As another example, the triage server 102 may generate new images relating to the patient (e.g., representative images of the patient's medical condition) to be stored in a server (e.g., a picture archiving and communication system (PACS) server) for retrieval by a physician. The new images may be based on the initial machine learning algorithm (and/or the selected machine learning algorithms). The triage server 102 may generate new metadata for use with the newly generated images. The generated new metadata may correct flaws with the original metadata (e.g., correct errors, add missing data, etc.). As another example, the triage server 102 may store a record in a server for retrieval by a physician. The record may include pixel data, metadata, patient information, results of preliminary machine learning algorithms, identification of selected machine learning algorithms, and resulting potential medical conditions.

In some instances, the image data may include multiple images and the acquisition condition may be a presence of an artifact in an image of the multiple images.

The systems, devices, and methods described herein result in an optimization of processing resources used to detect acquisition conditions. Acquisition conditions are identified on a per image basis using only the pixel data, which is then used to select the most appropriate selectable machine learning algorithms to determine medical conditions. This reduces of number of times multiple algorithms have to be executed relative to prior art systems in order to determine medical conditions, which is time consuming. As a result, the systems, devices, and methods described herein save time and generate relevant, accurate acquisition conditions.

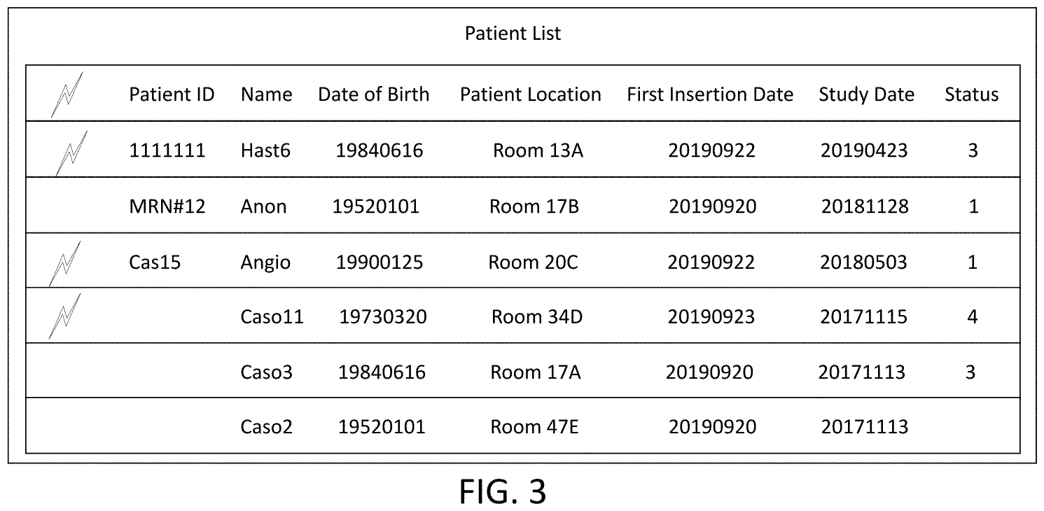

FIG. 3 shows an exemplary application that displays pop-up notifications of a patient list. The patient list may include, for each patient, a patient ID, patient name, patient date of birth, patient location, a first insertion date, a study date, status indicators, etc. The patient list may also include, for those patients with suspected predetermined medical conditions (e.g., ICH, LVO), an icon that indicates that the patient has a suspected predetermined medical condition. A compressed, small black and white image, generated based on the received image data, can be displayed as a preview function and marked as “not for diagnostic use.” This compressed preview is generally meant for informational purposes only, and does not contain any marking of the findings.

FIG. 4 shows an exemplary notification in the form of a pop-up containing a patient indicator, accession number and the type of suspected findings (e.g. ICH, LVO etc.). Presenting the physician with notifications can alert the physician of the need to quickly diagnose the patient with the suspected predetermined medical condition and, once diagnosis is confirmed, immediately provide appropriate treatment. Thus, the suspected condition receives attention earlier than would have been the case in the standard of care practice alone. The notifications may include an indication of how long ago the suspected medical condition has been detected to aid the physician in prioritizing patient care.

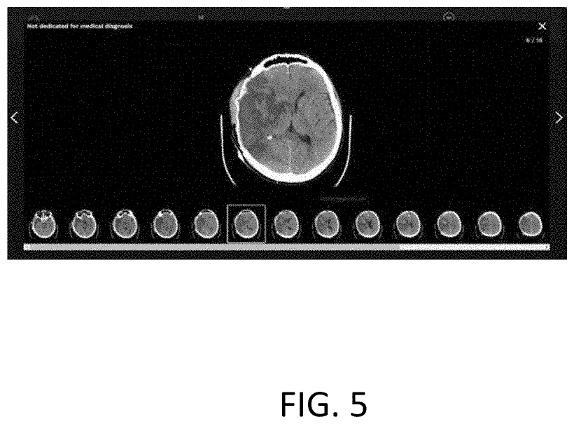

FIG. 5 shows exemplary image data (NCCT images) to which a machine learning algorithm can be applied by the triage server 102 to detect a medical condition (e.g. ICH). In an exemplary embodiment, a mask R-CNN algorithm can be used to identify and quantify image characteristics that are consistent with ICH. This provides a flexible and efficient framework for parallel evaluation of region proposal and object detection.

The triage device 102 can be used in a medical evaluation workflow, which can employ a wide variety of imaging data and other data representations (e.g. video/multi-image data) produced by various medical procedures and specialties. Such specialties include, but are not limited, to pathology, medical photography, medical data measurements such as electroencephalography (EEG) and electrocardiography (EKG) procedures, cardiology data, neuroscience data, preclinical imaging, and other data collection procedures occurring in connection with telemedicine, telepathology, remote diagnostics, and other applications of medical procedures and medical science.

A person having ordinary skill in the art would appreciate that embodiments of the disclosed subject matter (e.g. aforementioned triage server 102) can be practiced with various computer system configurations, including multi-core multiprocessor systems, minicomputers, mainframe computers, computers linked or clustered with distributed functions, as well as pervasive or miniature computers that can be embedded into virtually any device. For instance, one or more of the disclosed modules can be a hardware processor device with an associated memory.

A hardware processor device as discussed herein can be a single hardware processor, a plurality of hardware processors, or combinations thereof. Hardware processor devices can have one or more processor “cores.” The term “non-transitory computer readable medium” as discussed herein is used to generally refer to tangible media such as a memory device.

Various embodiments of the present disclosure are described in terms of an exemplary computing device. After reading this description, it will become apparent to a person skilled in the relevant art how to implement the present disclosure using other computer systems and/or computer architectures. Although operations can be described as a sequential process, some of the operations can in fact be performed in parallel, concurrently, and/or in a distributed environment, and with program code stored locally or remotely for access by single or multi-processor machines. In addition, in some embodiments the order of operations can be rearranged without departing from the spirit of the disclosed subject matter.

A hardware processor, as used herein, can be a special purpose or a general purpose processor device. The hardware processor device can be connected to a communications infrastructure, such as a bus, message queue, network, multi-core message-passing scheme, etc. An exemplary computing device, as used herein, can also include a memory (e.g., random access memory, read-only memory, etc.), and can also include one or more additional memories. The memory and the one or more additional memories can be read from and/or written to in a well-known manner. In an embodiment, the memory and the one or more additional memories can be non-transitory computer readable recording media.

Data stored in the exemplary computing device (e.g., in the memory) can be stored on any type of suitable computer readable media, such as optical storage (e.g., a compact disc, digital versatile disc, Blu-ray disc, etc.), magnetic storage (e.g., a hard disk drive), or solid-state drive. An operating system can be stored in the memory.

In an exemplary embodiment, the data can be configured in any type of suitable database configuration, such as a relational database, a structured query language (SQL) database, a distributed database, an object database, etc. Suitable configurations and storage types will be apparent to persons having skill in the relevant art.

The exemplary computing device can also include a communications interface. The communications interface can be configured to allow software and data to be transferred between the computing device and external devices. Exemplary communications interfaces can include a modem, a network interface (e.g., an Ethernet card), a communications port, a PCMCIA slot and card, etc. Software and data transferred via the communications interface can be in the form of signals, which can be electronic, electromagnetic, optical, or other signals as will be apparent to persons having skill in the relevant art. The signals can travel via a communications path, which can be configured to carry the signals and can be implemented using wire, cable, fiber optics, a phone line, a cellular phone link, a radio frequency link, etc.

Memory semiconductors (e.g., DRAMs, etc.) can be means for providing software to the computing device. Computer programs (e.g., computer control logic) can be stored in the memory. Computer programs can also be received via the communications interface. Such computer programs, when executed, can enable computing device to implement the present methods as discussed herein. In particular, the computer programs stored on a non-transitory computer-readable medium, when executed, can enable hardware processor device to implement the methods discussed herein. Accordingly, such computer programs can represent controllers of the computing device.

Where the present disclosure is implemented using software, the software can be stored in a computer program product or non-transitory computer readable medium and loaded into the computing device using a removable storage drive or communications interface. In an exemplary embodiment, any computing device disclosed herein can also include a display interface that outputs display signals to a display unit, e.g., LCD screen, plasma screen, LED screen, DLP screen, CRT screen, etc.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

What is claimed is:

1. A computing device comprising:
at least one processor; and
a memory, wherein the memory stores one or more instructions that, when executed by the processor, cause the computing device to:
receive image data of a patient and metadata associated with the image data;
analyze pixel information of the image data using a particular machine learning algorithm to determine one or more acquisition conditions;
select, based on the determined one or more acquisition conditions, one or more machine learning algorithms, from a set of machine learning algorithms, for analyzing the image data and the metadata to determine one or more medical conditions, wherein the particular machine learning algorithm is different from each machine learning algorithm of the set of machine learning algorithms.

2. The computing device of claim 1, wherein the one or more acquisition conditions are determined independent of the metadata.

3. The computing device of claim 1, wherein the one or more acquisition conditions includes a presence of at least a part of a particular organ in the image data.

4. The computing device of claim 1, wherein the image data comprises a plurality of images, wherein the one or more acquisition conditions includes a presence of an artifact in an image of the plurality of images.

5. The computing device of claim 1, wherein the one or more instructions, when executed by the processor, further cause the computing device to:
determine a priority for the patient based on a determination of the one or more medical conditions; and
update a worklist prioritizing patients based on the determined priority for the patient.

6. The computing device of claim 1, wherein the one or more acquisition conditions comprises a type of reconstruction.

7. The computing device of claim 1, wherein the one or more acquisition conditions comprises presence of contrast or contrast phase in imaged blood vessels in the image data.

8. The computing device of claim 1, wherein the one or more acquisition conditions comprises pre-surgery or post-surgery.

9. The computing device of claim 1, wherein the particular machine learning algorithm to determine the one or more acquisitions conditions is one of classification algorithms or segmentation algorithms based on convolution neuronal networks.

10. The computing device of claim 1, wherein the set of machine learning algorithms comprises one or more of automatic segmentation of a particular bodily organ, volume computation, intracranial hemorrhage detection, midline shift detection, hydrocephalus evaluation, large vessel occlusion detection, pulmonary embolism detection, and automatic stenosis evaluation.

11. The computing device of claim 1, wherein the instructions, when executed, further cause the computing device to send, to another computing device associated with a physician, a notification of the determined one or more medical conditions of the patient, the image data, and the metadata.

12. The computing device of claim 1, wherein the particular machine learning algorithm, when executed, causes:
generation of new images different from the received image data; and
storage of the generated new images for retrieval by a physician.

13. A method comprising:
receiving, by a computing device, image data of a patient and metadata associated with the image data;
analyzing, by the computing device, pixel information of the image data using a particular machine learning algorithm to determine one or more acquisition conditions;
selecting, by the computing device and based on the determined one or more acquisition conditions, one or more machine learning algorithms, from a set of machine learning algorithms, for analyzing the image data and the metadata to determine one or more medical conditions, wherein the particular machine learning algorithm is different from each machine learning algorithm of the set of machine learning algorithms.

14. The method of claim 13, wherein the one or more acquisition conditions are determined independent of the metadata.

15. The method of claim 13, wherein the one or more acquisition conditions includes a presence of at least a part of a particular organ in the image data.

16. The method of claim 13, wherein the image data comprises a plurality of images, wherein the one or more acquisition conditions includes a presence of an artifact in an image of the plurality of images.

17. The method of claim 13, further comprising:
determining a priority for the patient based on a determination of the one or more medical conditions; and
updating a worklist prioritizing patients based on the determined priority for the patient.

18. The method of claim 13, wherein the one or more acquisition conditions comprises a type of reconstruction.

19. The method of claim 13, wherein the one or more acquisition conditions comprises presence of contrast or contrast phase in imaged blood vessels in the image data.

20. The method of claim 13, wherein the one or more acquisition conditions comprises pre-surgery or post-surgery.

21. The method of claim 13, wherein the particular machine learning algorithm to determine the one or more acquisitions conditions is one of classification algorithms or segmentation algorithms based on convolution neuronal networks.

22. The method of claim 13, wherein the set of machine learning algorithms comprises one or more of automatic segmentation of a particular bodily organ, volume computation, intracranial hemorrhage detection, midline shift detection, hydrocephalus evaluation, large vessel occlusion detection, pulmonary embolism detection, and automatic stenosis evaluation.

23. The method of claim 13, further comprising:
sending, to another computing device associated with a physician, a notification of the determined one or more medical conditions of the patient, the image data, and the metadata.

24. The method of claim 13, further comprising:
generating new images different from the received image data based on the particular machine learning algorithm; and
storing the generated new images in a server for retrieval by a physician.
